(19) United States
(12) Patent Application Publication
(10) Pub. No.: US 2013/0195784 A1
(43) Pub. Date: Aug. 1, 2013

(54) WATER DISPERSIBLE SOLID DISPERSION OF PIGMENT

(71) Applicants: Michael Willis, Cincinnati, OH (US); Mike Jaffe, Cincinnati, OH (US); Jeff Carpenter, Loveland, OH (US); Colin Campbell, Loveland, OH (US); Judith Pharo, Hamilton, OH (US)

(72) Inventors: Michael Willis, Cincinnati, OH (US); Mike Jaffe, Cincinnati, OH (US); Jeff Carpenter, Loveland, OH (US); Colin Campbell, Loveland, OH (US); Judith Pharo, Hamilton, OH (US)

(73) Assignee: SUN CHEMICAL CORPORATION, Parsippany, NJ (US)

(21) Appl. No.: 13/878,198

(22) PCT Filed: Oct. 13, 2011
(86) PCT No.: PCT/US2011/056125

§ 371 Date: Apr. 5, 2013

Related U.S. Application Data
(60) Provisional application No. 61/392,509, filed on Oct. 13, 2010.

(51) Int. Cl. C08K 5/05; A61Q 5/02; A61Q 1/02; A61K 8/04; A61Q 1/10

(52) U.S. Cl. CPC C08K 5/05; A61K 8/04; A61Q 1/10; A61Q 1/02; A61Q 5/02
USPC 424/63; 424/70.7; 106/499; 106/501.1; 524/376; 106/31.86; 510/119; 510/513

(57) ABSTRACT

A solid dispersion of pigment for an aqueous composition includes a pigment and an ethoxylated alcohol. The solid dispersion of pigment is adapted for being directly incorporated into the aqueous composition with reduced shear for coloration. A method of preparing a solid dispersion of pigment for an aqueous composition with reduced shear mixing includes heating the mixture of an ethoxylated alcohol and a pigment, and cooling the mixture to obtain the solid dispersion of pigment.

This application claims the benefit of U.S. Provisional Patent Application No. 61/392,509, filed on Oct. 13, 2010, which is hereby incorporated by reference for all purposes as if fully set forth herein.

### FIELD OF INVENTION

The present inventions relate a solid dispersion of pigment for an aqueous composition, the solid dispersion of pigment including a pigment and an ethoxylated alcohol, and a method of preparing the solid dispersion of pigment.

### BACKGROUND OF THE INVENTION

Pigments are solid particles insoluble in the medium to which they are applied for the purpose of coloration in compositions, such as cosmetics, plastics, inks, paints and the like. Pigments can also be employed to provide functionality, such as brand identification, covert marking and indications of specific environmental exposures. Incorporation of pigments into any desired system requires mechanical and/or chemical energies to provide uniform color distribution to which pigment particle dispersion and particle stabilization is required.

Mechanical energies are applied in the form of shear and/or grinding and include, for example, attrition, extrusion, bead milling, high-speed stirring and 3-roll milling. Shear and grinding provide pigment comminution and/or deaggregation and deagglomeration, maximize pigment surface area exposure, provide uniform pigment distribution throughout the desired system, and thus yield best utilization of pigment value, such as color appearance or specific wavelength band interaction. Chemical energies, commonly achieved through dispersing agents, for example, surfactants, resins and polymeric dispersants, provide modifications at the pigment surface to reduce interfacial tension and allow for increased pigment surface wetting and pigment stabilization via ionic or steric hindrances or both. Efficacies of dispersing agents are dependent upon several factors, for example, physisorption, system compatibility and quantities used. Pigments treated with surfactants, resins or hyperdispersants are generally regarded as surface treated.

Pigments are available in various forms, for example, dry powder, presscake, fluid dispersion and solid dispersions, and may have been pre-processed to provide a stir-in form that does not require additional mechanical or chemical energies unless otherwise needed for specific application purposes. Generally, dry powder pigments and pigment presscakes require mechanical energies for proper dispersion into the applied system. Fluid dispersions and solid dispersions have already been dispersed and are generally used “as-is” without further mechanical energies other than mixing, dilution or dissolving into the desired system. A wide variety of dispersing agents for pigments are known in the art, yet they are not without limitations in applications, such as cosmetics and personal care products due to human health concerns.

Processing methods to produce a solid dispersion suitable for water phase applications typically include steps of preparing water phase dispersion. In these steps, water-soluble dispersants are adhered to the pigment surface. Water phase dispersions are prone to microbiological growth and typically require biocides to suppress and prevent microbial population. The biocides have limitations in end use applications, such as cosmetics and other personal care products due to health concerns. For example, U.S. Pat. No. 7,198,667, U.S. Pat. No. 5,648,408, and U.S. Pat. No. 5,820,666 disclose the use of conditioned organic pigments as stir-in pigments in various applications. The conditioned pigments were prepared by wet-milling a pigment crude.

Accordingly, there is a need to develop pigment in the form of water-soluble solid dispersion of pigment. The solid dispersion of pigment can be incorporated into an aqueous composition for coloration with reduced mixing. The solid dispersion of pigment does not include water, reduces the potential for microbiological growth in the aqueous solution, and eliminates the need for biocides.

### SUMMARY OF THE INVENTION

An advantage of the present inventions is to provide a solid dispersion of pigment for an aqueous composition. The solid dispersion may include a pigment and an ethoxylated alcohol, and the solid dispersion of pigment may be directly incorporated into the aqueous composition for coloration. The ethoxylated alcohol may have a carbon chain length of 20-50 and at least 3 molar equivalents of ethylene oxide, may have a melting point of between 40° C. to 120° C. or between 60° C. to 110° C., and may have a molecular weight of between 500 to 5,000 and an HLB value of between 4 to 20.

The solid dispersion of pigment may further include a resin, which is selected the group consisting of polyvinyl alcohol, polyvinylpyrrolidone, polyethylene glycol, and starches. The pigment may be selected from the group consisting of inorganic pigments, inorganic flakes, organic pigments, inorganic dye, and organic dye. The pigment may be about 5 wt % to 95 wt %, 25 wt % to 65 wt %, or 50 wt % based on the weight of the solid dispersion of pigment, and the ethoxylated alcohol may be about 5 wt % to 95 wt %, 35 wt % to 75 wt %, or 50 wt % based on the weight of the solid dispersion of pigment.

Another advantage of the present inventions is to provide a method of preparing a solid dispersion of pigment for an aqueous composition. The method may include heating a mixture of an ethoxylated alcohol and a pigment and cooling the mixture to obtain the solid dispersion of pigment. Heating the mixture of the ethoxylated alcohol and the pigment may include heating the ethoxylated alcohol until melted, adding the pigment to the melted ethoxylated alcohol, and mixing the pigment with the melted ethoxylated alcohol.

Yet another advantage of the present inventions is to provide a cosmetic or personal care product including the solid dispersion of pigment, an ink-jet ink including the solid dispersion of pigment, a coating including the solid dispersion of pigment, a fluid display device including the solid dispersion of pigment, or a marking device including the solid dispersion of pigment.

Yet another advantage of the present inventions is to provide a solid dispersion of pigment prepared according to the method described in this inventions.

Yet another advantage of the present inventions is to provide a method for coloring an aqueous composition without any high-shear mixing. The method includes preparing a solid dispersion of pigment according to the method described in this inventions, and applying the solid dispersion of pigment to the aqueous composition with reduced shear mixing.

Yet another advantage of the present inventions is to provide an aqueous composition colored from the solid dispersion of pigment prepared according to the method described in this inventions. The solid dispersion of pigment may be directly incorporated into the aqueous composition with reduced shear mixing.

Yet another advantage of the present inventions is to provide a solid dispersion of pigment for an aqueous composition. The solid dispersion of pigment includes a pigment and a resin, and eliminates the need for a step of preparing a water dispersion and the need for biocides. The solid dispersion of pigment also eliminates dusting hazard when incorporated into the aqueous composition.

Yet another advantage of the present inventions is to provide a solid dispersion of pigment for an aqueous composition. The solid dispersion of pigment includes a pigment and a resin, and is directly incorporated into the aqueous composition with reduced shear mixing for coloration.

It is to be understood that both the foregoing general description and the following detailed description are exemplary and explanatory and are intended to provide further explanation of the inventions as claimed.

### BRIEF DESCRIPTION OF THE DRAWINGS

The accompanying drawings, which are included to provide a further understanding of the inventions and are incorporated in and constitutes a part of this specification, illustrate embodiments of the inventions and together with the description serve to explain the principles of the inventions.

FIG. 1 shows the reflectance of Example 14 and Comparative Example 3 measured by a spectrophotometer.

FIG. 2 shows the reflectance of Example 15 and Comparative Example 3 measured by a spectrophotometer.

### DETAILED DESCRIPTION

Reference will now be made in detail to embodiments of the present inventions, example of which is illustrated in the accompanying drawing.

The inventors discovered that a water dispersible solid dispersion of one or more pigments and an ethoxylated alcohol is suitable for incorporating directly (by reduced shear stirring or mixing) into various aqueous compositions, such as, cosmetics, personal care products, ink jet inks, inks, paints, agricultural products (e.g. fertilizer and seed coatings), fluid display devices, liquid colorants and marking devices.

The term “solid dispersion of pigment” used herein refers to the dispersion of one or more pigments in a resin prepared by a melting, solvent, or melting-solvent method. The resin can be, for example, ethoxylated alcohols.

Ethoxylated alcohols (also called alcohol ethoxylates) are non-ionic surfactants. The ethoxylated alcohols in the present inventions may have the following general formula: R(OC₂H₄)nOH. The R group is a linear or branched alkyl group, an aryl group, or a heteroaryl group, and n is an integer. Preferably, the R group is a linear or branched alkyl group having 20 to 50 carbons, and n is an integer greater than or equal to 3.

In one embodiment, the ethoxylated alcohols have a carbon chain length of 20-50 and at least 3 molar equivalents of ethylene oxide. The melting point of the ethoxylated alcohols is between 40 and 120° C., preferably between 60 and 110° C. The molecular weight of the ethoxylated alcohols is between 500 to 5,000. The hydrophilic-lipophilic balance (HLB) value of the ethoxylated alcohols is between 4 and 20, preferably, between 10-18.

Other resin types could be used alone or in combination with the ethoxylated alcohol. Examples of other resin types include, but are not limited to polyvinyl alcohol, polyvinylpyrrolidone, polyethylene glycol and starches. It is understood that some of these materials may have a higher melt point than the 40-120° C. range, in which case a higher temperature may be required during the preparation of the solid dispersion of pigment.

Any ethoxylated alcohols falling within the definition above may be used in the present inventions. A number of such ethoxylated alcohols are available commercially. One example is the ethoxylated alcohols sold under the trademark Performathox™ from New Phase Technologies. Table 1 illustrates some of the Performathox™ products that are useful in preparing the solid dispersion of pigment of the inventions:

TABLE 1
Performathox ™ Ethoxylates

| | | Melt | Hydroxyl | Ethoxylate | |
|---|---|---|---|---|---|
| | | Point ° C. | Number | Content | |
| Product | INCI | USP XX | ASTM E-222 | NMR | HLB |
| TEST METHOD | Name | mod. | mod. | Calculation | Calculation |
| PERFORMATHOX ™ 420 | C₂₀₋₄₀ Pareth-3 | 90 | 80 | 20 | 4 |
| Ethoxylate | | | | | |
| PERFORMATHOX ™ 450 | C₂₀₋₄₀ Pareth-10 | 90 | 50 | 50 | 10 |
| Ethoxylate | | | | | |
| PERFORMATHOX ™ 480 | C₂₀₋₄₀ Pareth-40 | 88 | 20 | 80 | 16 |
| Ethoxylate | | | | | |
| PERFORMATHOX ™ 490 | Polyethylene | 70 | 10 | 90 | 18 |
| Ethoxylate | and C₂₀₋₄₀ | | | | |
| | Pareth-95 | | | | |

Another example of the suitable ethoxylated alcohols is UNITHOX™ Ethoxylates from Baker Hughes Incorporated. Table 2 illustrates some of the UNITHOX™ Ethoxylates products that are useful in preparing the solid dispersion of pigment of the inventions:

TABLE 2
UNITHOX ™ Ethoxylates

| | Molecular | Ethylene Oxide | | Hydroxyl Number | Melting Point |
|---|---|---|---|---|---|
| PRODUCT | Weight | (% by wt) | HLB Value | (mg KOH g sample) | (° C.) |
| Test Method | Calculation | NMR Calculation | Calculation | ASTM E-222 | ASTM D-127 |
| UNITHOX 420 Ethoxylate | 575 | 20 | 4 | 85 | 91 |
| UNITHOX 450 Ethoxylate | 920 | 50 | 10 | 55 | 91 |
| UNITHOX 480 Ethoxylate | 2300 | 80 | 16 | 22 | 86 |
| UNITHOX 490 Ethoxylate | 4600 | 90 | 18 | 12 | 71 |
| UNITHOX 550 Ethoxylate | 1100 | 50 | 10 | 41 | 99 |
| UNITHOX 720 Ethoxylate | 875 | 20 | 4 | 52 | 106 |
| UNITHOX 750 Ethoxylate | 1400 | 50 | 10 | 33 | 106 |

The pigments in the present inventions include inorganic pigments, high aspect ratio inorganic flakes, organic pigments, organic dyes, and inorganic dyes.

Suitable inorganic pigments include but are not limited to red iron oxide, yellow iron oxide, black iron oxide, brown iron oxide, titanium dioxide (rutile or anatase form), zinc oxide, zirconium oxide, cerium oxide, chromium oxide, chromium hydroxide, chromium hydrate, manganese violet, ferric ferrocyanide, magnesium carbonate, calcium carbonate, aluminum hydroxide, barium sulfate, ultramarine blues and combinations thereof.

High aspect ratio inorganic flakes may also be used either alone or in combination in the current inventions. Suitable examples include but are not limited to natural mica, synthetic mica, glass flakes, metal flakes, talc, kaolin, Al₂O₃ platelets, SiO₂ platelets, TiO₂ platelets, graphite platelets, BiOCl, calcium borosilicate and synthetic alumina. The substrates may be uncoated or coated with a metal oxide, such as TiO₂, Fe₂O₃, FeOOH, Fe₃O₄, ZrO₂, SnO₂, Cr₂O₃, BiOCl, and ZnO.

Suitable organic pigments include but are not limited to FD&C Blue #1, FD&C Yellow #5, FD&C Yellow #6, FD&C Yellow #7, FD&C Yellow #8, D&C Yellow #10, FD&C Red #2, FD&C Red #4, D&C Red #6, D&C Red #7, FD&C Red #9, FD&C Red #17, FD&C Red #19, D&C Red #21, D&C Red #22, D&C Red #27, D&C Red #28, D&C Red #30, FD&C Red #31, D&C Red #33, D&C Red #34, D&C Red #36, FD&C Red #40, D&C Violet #2, FD&C Green #3, D&C Green #5, D&C Green #6, D&C Green #8, D&C Orange #4, D&C Orange #5, D&C Brown #1, D&C Black #2, carmine quinacridone, anthraquinone, perylene, indigo, quinophthalone, indanthrone, isoindolinone, isoindoline, dioxazine, azo, phthalocyanine or diketopyrrolopyrrole series, Colour Index Pigment Yellow 1, Pigment Yellow 3, Pigment Yellow 12, Pigment Yellow 13, Pigment Yellow 14, Pigment Yellow 15, Pigment Yellow 62, Pigment Yellow 73, Pigment Yellow 74, Pigment Yellow 83, Pigment Yellow 93, Pigment Yellow 95, Pigment Yellow 109, Pigment Yellow 110, Pigment Yellow 111, Pigment Yellow 120, Pigment Yellow 128, Pigment Yellow 129, Pigment Yellow 139, Pigment Yellow 147, Pigment Yellow 151, Pigment Yellow 154, Pigment Yellow 168, Pigment Yellow 174, Pigment Yellow 175, Pigment Yellow 180, Pigment Yellow 181, Pigment Yellow 185, Pigment Yellow 188, Pigment Yellow 191:1, Pigment Yellow 194, Pigment Yellow 199, Pigment Orange 5, Pigment Orange 13, Pigment Orange 16, Pigment Orange 22, Pigment Orange 31, Pigment Orange 34, Pigment Orange 48, Pigment Orange 49, Pigment Orange 61, Pigment Orange 64, Pigment Orange 71, Pigment Orange 73, Pigment Red 2, Pigment Red 4, Pigment Red 5, Pigment Red 23, Pigment Red 42, Pigment Red 48:1, Pigment Red 48:2, Pigment Red 48:3, Pigment Red 48:4, Pigment Red 52:2, Pigment Red 53:1, Pigment Red 57:1, Pigment Red 112, Pigment Red 122, Pigment Red 144, Pigment Red 146, Pigment Red 166, Pigment Red 177, Pigment Red 184, Pigment Red 185, Pigment Red 202, Pigment Red 206, Pigment Red 214, Pigment Red 209, Pigment Red 220, Pigment Red 221, Pigment Red 222, Pigment Red 242, Pigment Red 248, Pigment Red 254, Pigment Red 255, Pigment Red 262, Pigment Red 264, Pigment Red 270, Pigment Red 272, Pigment Brown 23, Pigment Brown 25, Pigment Brown 41, Pigment Brown 42, Pigment Green 7, Pigment Green 36, Pigment Blue 15:1, Pigment Blue 15:2, Pigment Blue 15:3, Pigment Blue 15:6, Pigment Blue 16, Pigment Blue 25, Pigment Blue 26, Pigment Blue 29, Pigment Blue 60, Pigment Blue 64, Pigment Blue 66, Pigment Violet 19, Pigment Violet 23, Pigment Violet 29, Pigment Violet 31, Pigment Violet 32, Pigment Violet 37 and combinations thereof, including their lakes. Lakes include any counter-ion salt complex of the sulfonated species with a monovalent, divalent or trivalent atom or combinations thereof. Lakes also include said pigments made by extending on a substratum of clay, talc, blanc fixe, barium sulfate, titanium dioxide, zinc oxide, rosins, aluminum benzoate, alumina, aluminum hydrate, aluminum oxide, calcium carbonate or combinations thereof.

Other organic and inorganic pigments and dyes such as FD&C Red 30, FD&C Yellow 5, FD&C Yellow 6, FD&C Red 40, FD&C Blue 1, D&C Yellow 10, D&C Green 8, D&C Red 33, FD&C Green 3, Ext D&C Violet 2, D&C Orange 4 and Conchineal Red A, can also be employed, as well as combinations that achieve the colors desired, including their lakes as described above.

To prepare the solid dispersion of pigment of the present inventions, an ethoxylated alcohol is heated to a temperature close to its melting point. This temperature can be adjusted dependent upon the melt point and viscosity control. For example, the temperatures used in the process can be between 40 to 120° C., and more preferably between 60 to 110° C. Any conventional heating methods can be applied. After the ethoxylated alcohol is heated to a temperature close to its melting point, the ethoxylated alcohol is melted

A pigment or a combination of pigments are then added to the melted ethoxylated alcohol, and the mixture of the ethoxylated alcohol and the pigment is allowed to mix for an extended period of time. The period time depends upon the mixture, and can be between 1 to 12 hours. After mixing, the mixture is allowed to cool and solidify to obtain a solid dispersion of pigment.

It should be noted that the solid dispersion of pigment can be made without the use of a solvent slurry step and does not require biocide compounds. Various viscous mass mixing devices, such as an extruder, can be used. A preferred equipment for producing the solid dispersion is a sigma blade kneader.

In one embodiment, the solid dispersion of pigment includes about 5 to 95 wt % of pigment based on the weight of the solid dispersion of pigment and about 95 to 5 wt % of ethoxylated alcohol based on the weight of the solid dispersion of pigment. In another embodiment, the solid dispersion of pigment includes about 25 to 65 wt % of pigment based on the weight of the solid dispersion of pigment and about 75 to 35 wt % of ethoxylated alcohol based on the weight of the solid dispersion of pigment. In yet another embodiment, the solid dispersion of pigment includes about 50 wt % of pigment based on the weight of the solid dispersion of pigment and about 50 wt % of ethoxylated alcohol based on the weight of the solid dispersion of pigment.

For example, a method as described in Example 1 (below) involves heating a jacketed kneader by steam or hot oil circulation containing the ethoxylated alcohol to achieve an ethoxylated alcohol melt followed by dry pigment addition while the mixing blades are rotating. The mixture of the pigment and the ethoxylated alcohol is allowed to mix preferably for a minimum of 2 hours then allowed to cool with the mixing blades idle. The blades are engaged once the mixture has solidified, and the mixture is fragmented for unloading and final grinding to achieve a pulverized solid mixture of pigment dispersed into the ethoxylated alcohol. This method to produce the solid pigment dispersion results in a significant improvement in pigment loading resulting in a lesser impact of the resin (the ethoxylated alcohol) on the final product formulation.

Typical high shear energy and milling conditions required to disperse dry pigmented forms into fluid or paste bases utilize rotor stators, bead mills, Cowles blades, impingement methods and 3 roll mills for example. An advantage of the current inventive solid dispersions of pigment is that the solid dispersions of pigment can be directly incorporated into aqueous compositions by reduced shear mixing, eliminating the need for high-shear mixing. Reduced shear mixing used herein means simple mixing or stirring using shear energy substantively reduced from typical high shear energy.

Another advantage of the current inventive solid dispersions of pigment is that water dispersion steps are not required. Thus, it eliminates the need for costly and time consuming water removal, eliminates or greatly reduced the potential for microbiological growth, and eliminates the need for biocides.

Another advantage of the current inventive solid dispersions of pigment is their potentially lower dusting hazard compared to typical powder pigments when incorporated into end use compositions. This could enable the use of an expanded palate of pigments that may not normally be used due to dusting hazards.

### EXAMPLES

### Example 1

### Preparation of D&C Black 2 Solid Dispersion in Ethoxylated Alcohol

To a 1 gallon stainless steel sigma blade kneader was added 550 g Performathox™ 490 ethoxylated alcohol (INCI NAME: C20-C40 Pareth-95 supplied by NEW PHASE TECHNOLOGIES). Steam at approximately 122° C. was circulated through the jacket until the resin (ethoxylated alcohol) was melted. Mixing was initiated and 450 g D&C Black 2 (SunCROMA™ C47-2222, SunChemical) was added over 30 minutes. The mass was allowed to mix for 3 hours with continuous steam input to the kneader jacket. The sides of the kneader were scraped approximately every 30 minutes. After 3 hours of mixing, the steam and mixing blades were turned off to allow for the mass to cool and solidify, aided by the addition of dry ice. Once solidified, the blades were jogged to break loose the mass and pulverize the mass. The pulverized mass was then unloaded and a portion was ground into a course powdered solid using an Osterizer cup with cutting blades.

Example 2 - Ink Jet Ink

| Ink Compositions | wt % |
|---|---|
| Liponic EG1 (humectant) | 5.00 |
| 1,5-pentanediol | 5.00 |
| 2-pyrrolidinone | 5.00 |
| 2-methyl-2,4-pentanediol | 2.25 |
| 2-methyl-1,3-propanetriol | 5.00 |
| 3-methyl-1,3,5-pentanetriol | 3.50 |
| Surfynol 465 (surfactant) | 0.05 |
| triethanolamine | 0.25 |
| ammonium acetate | 1.00 |
| Proxel GXL (biocide) | 0.50 |
| Example 1 Dispersion | 11.00 |
| Deionized H2O | 61.45 |
| Total | 100.00 |

A pre-mixture was prepared with the Example 1 dispersion and deionized water. The pre-mixture was mixed and heated for 15 minutes. The pre-mixture was then allowed to cool to room temperature. To this pre-mixture was added additional ink formulation reagents while stirring with a Cowls blade. The ensuing mixture was then further processed using an Eiger mill for 60 min at 4500 rpm with 1.0 mm ceramic media to produce a finished ink with sufficient strength, viscosity and surface tension properties for use as a typical ink jet ink. It is worth noting that the Example 1 dispersion did not have a deleterious effect on surface tension after milling. Both before and after milling, the surface tension was measured at 44.4 dynes/cm using the Wilhelmy Plate Method.

Example 3 - Water Resistant Mascara

| Mascara Components | | wt % |
|---|---|---|
| Phase A | Water | 66.33 |
| | Hydrated Magnesium Aluminum Silicate Mineral | 1.11 |
| | Example 1 Dispersion | 11.11 |
| Phase B | Triethanolamine | 0.33 |
| | Propylene Glycol | 8.89 |
| | Xanthan Gum | 0.33 |
| | Phenoxyethanol (and) methyl-, ethyl-, propyl-, | 1.00 |
| | butylparaben | |
| Phase C | Stearic Acid | 4.44 |
| | Glyceril Stearate | 0.9 |
| | Oleyl Alcohol | 0.56 |
| | Beeswax (Cera Alba) | 3.00 |
| Phase D | Styrene/Acrylate/Ammonium Methacrylate | 2.00 |
| | Copolymer Emulsion | |
| Total | | 100.00 |

Process:

1. Heat water to 75° C. and add Hydrated Magnesium Aluminum Silicate Mineral under high-shear mixing. Mix for 15 minutes until Hydrated Magnesium Aluminum Silicate Mineral is fully hydrated. Then add Example 1 Dispersion under stirring while maintaining temperature ˜80° C. until fully dispersed.

2. Pre-disperse Xanthan Gum in Propylene Glycol and add to Phase A. Then, add remaining ingredients of Phase B into A maintaining same ˜80° C. temperature.

3. Heat Phase C to 80° C. and add it to Phases A/B under mixing Silverson high-shear mixer (L4RT type), speed at about 2000 rpm, increase to about 3000 rpm to homogenize the bulk, decreasing to about 1000 rpm to begin cooling, allow the bulk to homogenize for 5-10 minutes when cooling down.

4. Allow for cooling under slow agitation. At approximately 40° C., add Phase D with slow agitation. Once Phase D is incorporated, stop agitation and allow cooling to room temperature.

### Example 4

### Preparation of D&C Red 7 Solid Dispersion in Ethoxylated Alcohol

To a 1 gallon stainless steel sigma blade kneader was added 750 g Performathox™ 490 (INCI NAME: C20-C40 Pareth-95 supplied by NEW PHASE TECHNOLOGIES). Steam at approximately 122° C. was circulated through the jacket until the resin was melted. Mixing was initiated and 1800 g D&C Red 7 Ca Lake (SunCROMA™ C19-021, Sun Chemical) was added over 90 minutes. The mass was allowed to mix for 3 hours with continuous steam input to the kneader jacket. The sides of the kneader were scraped approximately every 30 minutes. After 3 hours of mixing, the steam and mixing blades were turned off to allow for the mass to cool and solidify. Once solidified, the blades were jogged to break loose the mass and pulverize the mass. The pulverized mass was then unloaded and a portion was ground in an Osterizer cup with cutting blades.

### Example 5

### Aqueous Seed Coating Colorant

To 50.75 parts by weight room temperature water is added 35 parts by weight Example 4 Dispersion, 5 parts by weight bentonite clay, 9 parts by weight propylene glycol, 0.2 parts by weight Proxel GXL and 0.05 parts by weight Surfynol 104. Stir for 1 hour at 50-55° C. using an overhead paddle stirrer.

### Example 6

### Preparation of D&C Orange 4 Solid Dispersion in Ethoxylated Alcohol

To a 0.5 oz container was added 3 g Performathox™ 490 (INCI NAME: C20-C40 Pareth-95 supplied by NEW PHASE TECHNOLOGIES) and 1 g SunCROMA™ C76-7393 D&C Orange 4. The container was sealed and the mixture was spun on a FlackTek SpeedMixer™ DAC 150 FV(Z) at 3300 rpm for 4 minutes. The resulting mass had melted and the resulting form was a solid colored dispersion.

### Example 7

### Shampoo

To 32 oz distilled water at 55° C. is added 0.01 g Example 6 Dispersion. Stir to fully dissolve the solid colorant. Allow to cool to room temperature and add 1¼ Cups Initial Soap Concentrate (Sodium Laureth Sulfate, cocamidopropyl Betaine, Cocamide DEA, Cocamidopropyl hydroxysultaine, Glydent Antimicrobial, EDTA-4NA organic chelating agent and 1.2 ounces humectant). Gently mix for 1-2 minutes. Allow mixture to sit overnight.

### Example 8

### Preparation of Red Iron Oxide Solid Dispersion in Ethoxylated Alcohol

To a 1 gallon stainless steel sigma blade kneader was added 650 g Performathox™ 490 (INCI NAME: C20-C40 Pareth-95 supplied by NEW PHASE TECHNOLOGIES). Steam at approximately 122° C. was circulated through the jacket until the resin was melted. Mixing was initiated and 1750 g Red Iron Oxide (SunCROMA™ C33-8075, Sun Chemical) was added over 60 minutes. The mass was allowed to mix for 3 hours with continuous steam input to the kneader jacket. The sides of the kneader were scraped approximately every 30 minutes. After 3 hours of mixing, the steam and mixing blades were turned off to allow for the mass to cool and solidify. Once solidified, the blades were jogged to break loose the mass and pulverize the mass. The pulverized mass was then unloaded and a portion was ground in an Osterizer cup with cutting blades.

### Example 9

### Interior Latex Paint

To an 8 oz jar containing 90.0 g Porter 939 is added 1.0 g Example 8 Dispersion. The lid is secured and the jar mixture is shook on a paint shaker for 60 minutes.

### Example 10

### Preparation of Brown Iron Oxide Solid Dispersion in Ethoxylated Alcohol

To a 1 gallon stainless steel sigma blade kneader was added 720 g Performathox™ 490 (INCI NAME: C20-C40 Pareth-95 supplied by NEW PHASE TECHNOLOGIES). Steam at approximately 122° C. was circulated through the jacket until the resin was melted. Mixing was initiated and 1680 g Brown Iron Oxide (SunCROMA™ C33-115, Sun Chemical) was added over 60 minutes. The mass was allowed to mix for 3 hours with continuous steam input to the kneader jacket. The sides of the kneader were scraped approximately every 30 minutes. After 3 hours of mixing, the steam and mixing blades were turned off to allow for the mass to cool and solidify. Once solidified, the blades were jogged to break loose the mass and pulverize the mass. The pulverized mass was then unloaded and a portion was ground in an Osterizer cup with cutting blades.

### Example 11

### Interior Latex Paint

To an 8 oz jar containing 90.0 g Porter 939 is added 1.0 g Example 10 Dispersion. The lid is secured and the jar mixture is shook on a paint shaker for 60 minutes.

### Example 12

### Cream Makeup

To a 1 oz plastic container was added 10.0 g water based white cream makeup (Collegeville Imagineering Ent.) and 0.07 g of EXAMPLE 10 Brown Ion Oxide Solid Dispersion in Ethoxylated Alcohol. The container was sealed and mixed at 3500 rpms for 2×3 minutes on a FlackTek, Inc. Speedmixer™. The container was removed and the colored mixture was mounted in a display card for spectrophotometric measurement.

### Comparative Example 1

### Cream Makeup

To a 1 oz plastic container was added 10.0 g water based white cream makeup (Collegeville Imagineering Ent.), 0.05 g SunCROMA™ C33-115 (Brown Ion Oxide), and 0.02 g Performathox® 490 ethoxylated alcohol. The container was sealed and mixed at 3500 rpms for 2×3 minutes on a FlackTek, Inc. Speedmixer™. The container was removed and the colored mixture was mounted in a display card for spectrophotometric measurement.

### Comparison of Example 12 and Comparative Example 1

Spectrophotomer readings (DataColor SF600: D65, 10 degrees, specular included) of Example 12 using Comparative Example 1 as standard are as follows: Example 12 is 118% stronger (K/S at WL 420) and 1.92 units yellower (Db −1.92) than Comparative Example 1. Spectrophotomer readings are recorded in Table 3.

TABLE 3
Spectrophotomer Readings of Example 12 Using
Comparative Example 1 as Standard

| WL of Max Abs | % R | K/S |
|---|---|---|
| 420 | 44.42 | 0.3478 |
| 420 | 41.62 | 0.4095 |

CIELab Color Difference

| | DE* | DL* | Da* | Db* | DC* | DH* |
|---|---|---|---|---|---|---|
| Unadjusted: | 2.25 | −0.62 | 0.98 | 1.92 | 2.03 | 0.73 |
| Adjusted Strength: | 1.71 | 0.93 | 0.40 | 1.37 | 1.23 | 0.73 |

FMC-II Color Difference

| | DE | DCRG | DCYB | DL | DC |
|---|---|---|---|---|---|
| Unadjusted: | 6.56 | 5.54 | 3.17 | −1.52 | 6.38 |
| Adjusted Strength: | 4.16 | 2.68 | 2.13 | 2.36 | 3.42 |

CMC Color Difference

| | DE | DL*/SL | DC*/SC | DH*/SH |
|---|---|---|---|---|
| Unadjusted: | 1.98 | −0.23 | 1.58 | 1.18 |
| Adjusted Strength: | 1.56 | 0.35 | 0.96 | 1.18 |

In Example 12, the Brown Ion Oxide solid dispersion was directly incorporated into the white cream makeup. The Brown Ion Oxide pigment was uniformly distributed in the white cream makeup, yielding best utilization of pigment value. In comparative Example 1, the Brown Ion Oxide pigment was aggregated, and not uniformly distributed in the white cream makeup.

### Example 13

### Cream Makeup

To a 1 oz plastic container was added 10.0 g water based white cream makeup (Collegeville Imagineering Ent.) and 0.07 g of EXAMPLE 4 D&C Red 7 Solid Dispersion in Ethoxylated Alcohol. The container was sealed and mixed at 3500 rpms for 2×3 minutes on a FlackTek, Inc. Speedmixer™. The container was removed and the colored mixture was mounted in a display card for spectrophotometric measurement.

### Comparative Example 2

### Cream Makeup

To a 1 oz plastic container was added 10.0 g water based white cream makeup (Collegeville Imagineering Ent.), 0.05 g SunCROMA™ C19-021 (D&C Red 7 Ca Lake), and 0.02 g Performathox® 490 ethoxylated alcohol. The container was sealed and mixed at 3500 rpms for 2×3 minutes on a FlackTek, Inc. Speedmixer™. The container was removed and the colored mixture was mounted in a display card for spectrophotometric measurement.

### Comparison of Example 13 and Comparative Example 2

Spectrophotomer readings (DataColor SF600: D65, 10 degrees, specular included) of Example 13 using Comparative Example 2 as standard are as follows: Example 13 is 157% stronger (K/S at WL 500) and 4.76 units redder (Da 4.76) than Comparative Example 2. Spectrophotomer readings are recorded in Table 4.

TABLE 4
Spectrophotomer Readings of Example 13 Using
Comparative Example 2 as Standard

| WL of Max Abs | % R | K/S |
|---|---|---|
| 500 | 47.39 | 0.2920 |
| 500 | 39.74 | 0.4570 |

CIELab Color Difference

| | DE* | DL* | Da* | Db* | DC* | DH* |
|---|---|---|---|---|---|---|
| Unadjusted: | 6.65 | −2.04 | 4.76 | 4.17 | 6.29 | 0.70 |
| Adjusted Strength: | 1.40 | 0.51 | 0.94 | 0.91 | 1.29 | 0.23 |

FMC-II Color Difference

| | DE | DCRG | DCYB | DL | DC |
|---|---|---|---|---|---|
| Unadjusted: | 21.52 | 19.77 | 7.12 | −4.64 | 21.02 |
| Adjusted Strength: | 4.06 | 3.54 | 1.44 | 1.37 | 3.82 |

CMC Color Difference

| | DE | DL*/SL | DC*/SC | DH*/SH |
|---|---|---|---|---|
| Unadjusted: | 3.68 | −0.73 | 3.52 | 0.78 |
| Adjusted Strength: | 0.78 | 0.18 | 0.72 | 0.25 |

In Example 13, the D&C Red 7 solid dispersion was directly incorporated into the white cream makeup. The D&C Red 7 pigment was uniformly distributed in the white cream makeup, yielding best utilization of pigment value. In comparative Example 2, the D&C Red 7 pigment was aggregated, and not uniformly distributed in the white cream makeup.

### Example 14

### Cream Makeup

To a 1 oz plastic container was added 10.0 g water based white cream makeup (Collegeville Imagineering Ent.) and 0.11 g of EXAMPLE 1 D&C Black 2 Solid Dispersion in Ethoxylated Alcohol (45% SunCROMA™ C47-2222 D&C Black 2 and 55% Performathox® 490 ethoxylated alcohol). The container was sealed and mixed at 3500 rpms for 2×3 minutes on a FlackTek, Inc. Speedmixer™. The container was removed and the colored mixture was mounted in a display card for spectrophotometric measurement.

### Example 15

### Cream Makeup

To a 1 oz plastic container was added 10.0 g water based white cream makeup (Collegeville Imagineering Ent.) and 0.11 g of EXAMPLE 1 D&C Black 2 Solid Dispersion in Ethoxylated Alcohol (45% SunCROMA™ C47-2222 D&C Black 2 and 55% Performathox® 490 ethoxylated alcohol). The container was sealed and mixed at 1000 rpms for 2×3 minutes on a FlackTek, Inc. Speedmixer™. The container was removed and the colored mixture was mounted in a display card for spectrophotometric measurement.

### Comparative Example 3

### Cream Makeup

To a 1 oz plastic container was added 10.0 g water based white cream makeup (Collegeville Imagineering Ent.), 0.05 g SunCROMA™ C47-2222 (D&C Black 2), and 0.06 g Performathox® 490 ethoxylated alcohol. The container was sealed and mixed at 3500 rpms for 2×3 minutes on a FlackTek, Inc. Speedmixer™. The container was removed and the colored mixture was mounted in a display card for spectrophotometric measurement.

### Comparison of Examples 14 and 15 and Comparative Example 3

Visually Comparative Example 3 was light gray with little coloration from the black pigment. Examples 14 and 15 were visually darker and a grey dispersion as intended.

Spectrophotomer readings (DataColor SF600: D65, 10 degrees, specular included) of Example 14 using Comparative Example 3 as standard are as follows: Example 14 is 2484.8% stronger (K/S at WL 680) and 39.67 units darker (DL −39.67) than Comparative Example 3. Spectrophotomer readings are recorded in Table 5.

TABLE 5
Spectrophotomer Readings of Example 14 Using
Comparative Example 3 as Standard

| WL of Max Abs | % R | K/S |
|---|---|---|
| 680 | 63.48 | 0.1051 |
| 680 | 14.12 | 2.6107 |

CIELab Color Difference

| | DE* | DL* | Da* | Db* | DC* | DH* |
|---|---|---|---|---|---|---|
| Unadjusted: | 39.67 | −39.67 | 0.19 | 0.49 | −0.52 | 0.02 |
| Adjusted Strength: | 1.78 | −1.04 | 0.47 | 1.36 | −1.44 | 0.00 |

FMC-II Color Difference

| | DE | DCRG | DCYB | DL | DC |
|---|---|---|---|---|---|
| Unadjusted: | 66.13 | −0.29 | −0.21 | −66.13 | 0.36 |
| Adjusted Strength: | 4.62 | 3.18 | 2.07 | −2.63 | 3.80 |

CMC Color Difference

| | DE | DL*/SL | DC*/SC | DH*/SH |
|---|---|---|---|---|
| Unadjusted: | 14.23 | −14.21 | −0.67 | 0.02 |
| Adjusted Strength: | 1.89 | −0.37 | −1.85 | 0.00 |

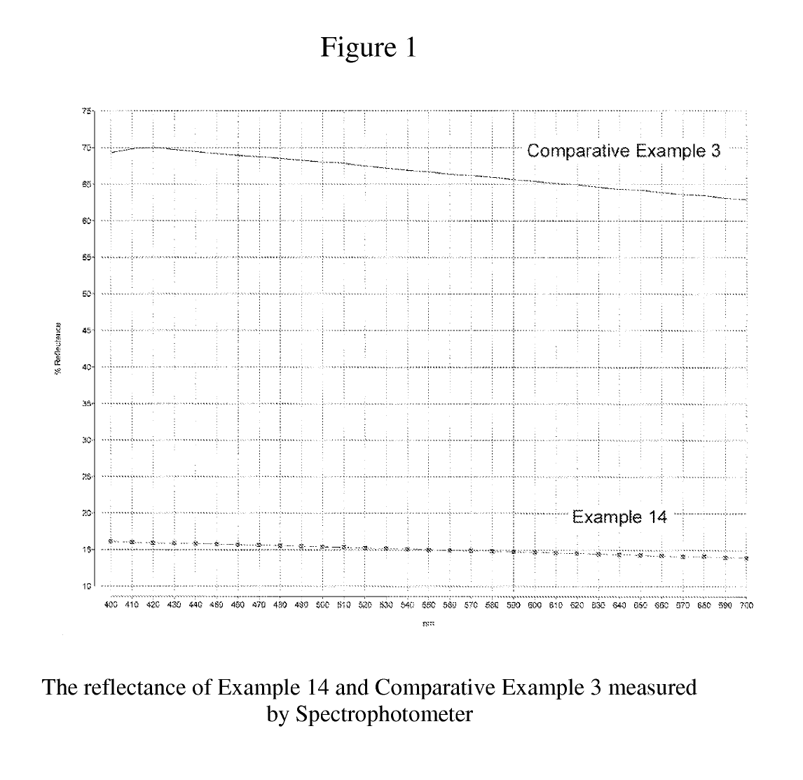

The reflectance of Example 14 and Comparative Example 3 is shown in FIG. 1. The Spectral Response Curve in FIG. 1 demonstrates that Comparative Example 3 has higher reflectance across 400-700 nm wavelengths than Example 14.

Spectrophotomer readings of Example 15 (DataColor SF600: D65, 10 degrees, specular included) using Comparative Example 3 as standard are as follows: Example 15 is 1939.6% stronger (K/S at WL 680) and 36.36 units darker (DL −36.36) than Comparative Example 3. Spectrophotomer readings are recorded in Table 6.

TABLE 6
Spectrophotomer Readings of Example 15 Using
Comparative Example 3 as Standard

| WL of Max Abs | % R | K/S |
|---|---|---|
| 680 | 63.48 | 0.1051 |
| 680 | 16.93 | 2.0385 |

CIELab Color Difference

| | DE* | DL* | Da* | Db* | DC* | DH* |
|---|---|---|---|---|---|---|
| Unadjusted: | 36.38 | −36.36 | 0.35 | 1.22 | −1.27 | −0.09 |
| Adjusted Strength: | 2.18 | −1.26 | 0.55 | 1.70 | −1.78 | −0.06 |

FMC-II Color Difference

| | DE | DCRG | DCYB | DL | DC |
|---|---|---|---|---|---|
| Unadjusted: | 62.87 | 0.38 | 0.30 | −62.87 | 0.49 |
| Adjusted Strength: | 5.62 | 3.87 | 2.57 | −3.16 | 4.64 |

CMC Color Difference

| | DE | DL*/SL | DC*/SC | DH*/SH |
|---|---|---|---|---|
| Unadjusted: | 13.13 | −13.03 | −1.63 | −0.13 |
| Adjusted Strength: | 2.34 | −0.45 | −2.29 | −0.08 |

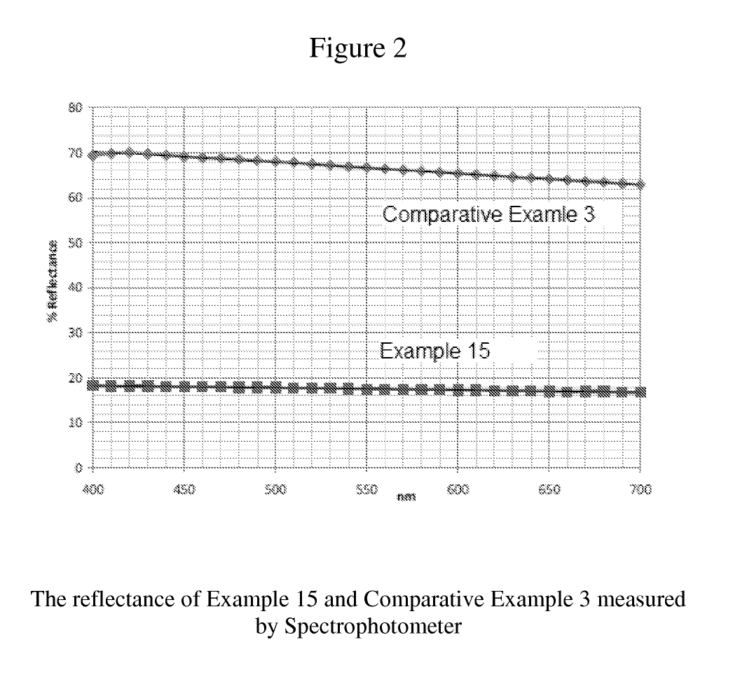

The reflectance of Example 15 and Comparative Example 3 is shown in FIG. 2. The Spectral Response Curve in FIG. 2 demonstrates that Comparative Example 3 has higher reflectance across 400-700 nm wavelengths than Example 15.

In Examples 14 and 15, the D&C Black 2 solid dispersion was directly incorporated into the white cream makeup, and the D&C Black 2 pigment was uniformly distributed in the white cream makeup, yielding best utilization of pigment value. In comparative Example 3, the D&C Black 2 pigment was aggregated, and not uniformly distributed in the white cream makeup.

While both Examples 14 and 15 obtain uniform distribution of pigment and yield best utilization of pigment value, the mixing in Example 14 (3500 rpms for 2×3 minutes on a FlackTek, Inc. Speedmixer™) uses much higher shear energy than the mixing in Example 15 (1000 rpms for 2×3 minutes on a FlackTek, Inc. Speedmixer™). Therefore, it establishes that the D&C Black 2 solid dispersion can be directly incorporated into aqueous composition with reduced shear mixing.

It will be apparent to those skilled in the art that various modifications and variations can be made in the present inventions without departing from the spirit or scope of the inventions. Thus, it is intended that the present inventions cover the modifications and variations of this inventions provided they come within the scope of the appended claims and their equivalents.

1. A solid dispersion of pigment comprising:
a pigment present in an amount of from about 5 wt % and 95 wt % based on the weight of the dispersion; and
an ethoxylated alcohol present in an amount of from about 5 wt % and 95 wt % based on the weight of the dispersion, wherein:
the solid dispersion of pigment directly incorporates into an aqueous composition.

2. The solid dispersion of pigment of claim 1, wherein the ethoxylated alcohol has a carbon chain length of 20-50 and at least 3 molar equivalents of ethylene oxide, a melting point of between 40° C. to 120° C., a molecular weight of between 500 to 5,000 and an HLB value of between 4 to 20.

3. The solid dispersion of pigment of claim 1, wherein the ethoxylated alcohol has a melting point of between 60° C. to 110° C.

4. (canceled)

5. The solid dispersion of pigment of claim 1 further comprising a resin.

6. The solid dispersion of pigment of claim 5, wherein the resin is selected from among polyvinyl alcohol, polyvinylpyrrolidone, polyethylene glycol, and starches.

7. The solid dispersion of pigment of claim 1, wherein the pigment is selected from among inorganic pigments, inorganic flakes, organic pigments, inorganic dyes, and organic dyes.

8. The solid dispersion of pigment of claim 1, wherein the pigment is present in an amount of from between 25 wt % to 65 wt % based on the weight of the solid dispersion of pigment.

9. A method of preparing the solid dispersion of pigment of claim 1 comprising:
heating a mixture of an ethoxylated alcohol and a pigment, and
cooling the mixture to obtain the solid dispersion of pigment.

10. The method of claim 9, wherein heating the mixture of the ethoxylated alcohol and the pigment comprises:
heating the ethoxylated alcohol until melted;
adding the pigment to the melted ethoxylated alcohol; and
mixing the pigment with the melted ethoxylated alcohol.

11. The method of claim 9, wherein the ethoxylated alcohol has a carbon chain length of 20-50 and at least 3 molar equivalents of ethylene oxide, a melting point of between 40° C. to 120° C., a molecular weight of between 500 to 5,000 and an HLB value of between 4 to 20.

12. The method of claim 9, wherein the ethoxylated alcohol has a melting point of between 60° C. to 110° C.

13. (canceled)

14. The method of claim 9 further comprising adding a resin to the mixture of the ethoxylated alcohol and the pigment.

15. The method of claim 14, wherein the resin is selected from among polyvinyl alcohol, polyvinylpyrrolidone, polyethylene glycol, and starches.

16. The method of claim 9, wherein the pigment is selected from among inorganic pigments, inorganic flakes, organic pigments, inorganic dyes, and organic dyes.

17. The method of claim 9, wherein:
the pigment is present in an amount of from about 5 wt % to 95 wt %, based on the weight of the solid dispersion of pigment; and
the ethoxylated alcohol is present in an amount of from about 5 wt % to 95 wt %, based on the weight of the solid dispersion of pigment.

18. A cosmetic or personal care product comprising the solid dispersion of pigment of claim 1.

19. An ink jet ink comprising the solid dispersion of pigment of claim 1.

20. A coating comprising the solid dispersion of pigment of claim 1.

21. A fluid display device comprising the solid dispersion of pigment of claim 1.

22. A marking device comprising the solid dispersion of pigment of claim 1.

23. (canceled)

24. The method of claim 9, further comprising incorporating the solid dispersion of pigment into an aqueous composition with reduced shear mixing.

25. An aqueous composition comprising the solid dispersion of pigment prepared according to the method of claim 9.

26. The solid dispersion of pigment of claim 1 that is free of biocides.

27. The solid dispersion of pigment of claim 1, wherein the solid dispersion of pigment does not create a dusting hazard when incorporated into an aqueous composition.

28. (canceled)
